(19) United States
(12) Patent Application Publication
(10) Pub. No.: US 2022/0392626 A1
(43) Pub. Date: Dec. 8, 2022

(54) BODY DYNAMICS SYSTEM

(71) Applicant: Victor Aarre, Stavanger (NO)

(72) Inventor: Victor Aarre, Stavanger (NO)

(21) Appl. No.: 17/775,360

(22) PCT Filed: Nov. 9, 2020
(86) PCT No.: PCT/IB2020/060536

§ 371 Date: May 9, 2022

Related U.S. Application Data
(60) Provisional application No. 62/933,479, filed on Nov. 10, 2019.

(51) Int. Cl. G16H 40/63; G06N 5/02

(52) U.S. Cl. CPC G16H 40/63; G06N 5/022

(57) ABSTRACT

A method can include receiving data for a body where the data include energy consumption data and weight change data; generating an efficiency parameter value using the received data, where the efficiency parameter depends on a difference of an amount of water in two different types of body tissues of the body; and generating an output using the efficiency parameter, where such a method can be performed via circuitry of a system or a device, which may include a weight scale, a wearable computing device or a smart phone.

### RELATED APPLICATIONS

This application is a 35 USC 371 application for entry of the national phase in the US that claims priority to and the benefit of a pending Patent Cooperation Treaty application having International Application No. PCT/IB2020/060536 with an International Filing Date of 9 Nov. 2020 (published as WO 2021/090296 A1 on 14 May 2021), which is incorporated by reference herein and which claims priority to and the benefit of a US Provisional Application having Ser. No. 62/933,479, filed 10 Nov. 2019, which is incorporated by reference herein.

### BACKGROUND

A body can vary over time depending on consumption of material by the body and activity of the body. Body dynamics can include loss of weight or gain of weight.

### SUMMARY

A method can include receiving data for a body where the data include energy consumption data and weight change data; generating an efficiency parameter value using the received data, where the efficiency parameter depends on a difference of an amount of water in two different types of body tissues of the body; and generating an output using the efficiency parameter. Various other apparatuses, systems, methods, etc., are also disclosed. This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

Features and advantages of the described implementations can be more readily understood by reference to the following description taken in conjunction with the accompanying drawings.

FIG. 1 illustrates an example of a system, examples of devices and examples of environments;

FIG. 2 illustrates an example of a method and an example of equipment;

FIG. 3 illustrates an example of a method;

FIG. 4 illustrates an example of a system;

FIG. 5 illustrates an example of a method with respect to examples of graphical user interfaces (GUIs);

FIG. 6 illustrates an example of a graphical user interface (GUI);

FIG. 7 illustrates an example of a graphical user interface (GUI);

FIG. 8 illustrates an example of a graphical user interface (GUI);

FIG. 9 illustrates an example of a graphical user interface (GUI);

FIG. 10 illustrates an example of a graphical user interface (GUI);

FIG. 11 illustrates an example of a graphical user interface (GUI);

FIG. 12 illustrates examples of system components and an example of a method; and

FIG. 13 illustrates example components of a system and a networked system.

### DETAILED DESCRIPTION

This description is not to be taken in a limiting sense, but rather is made merely for the purpose of describing the general principles of the implementations. The scope of the described implementations should be ascertained with reference to the issued claims.

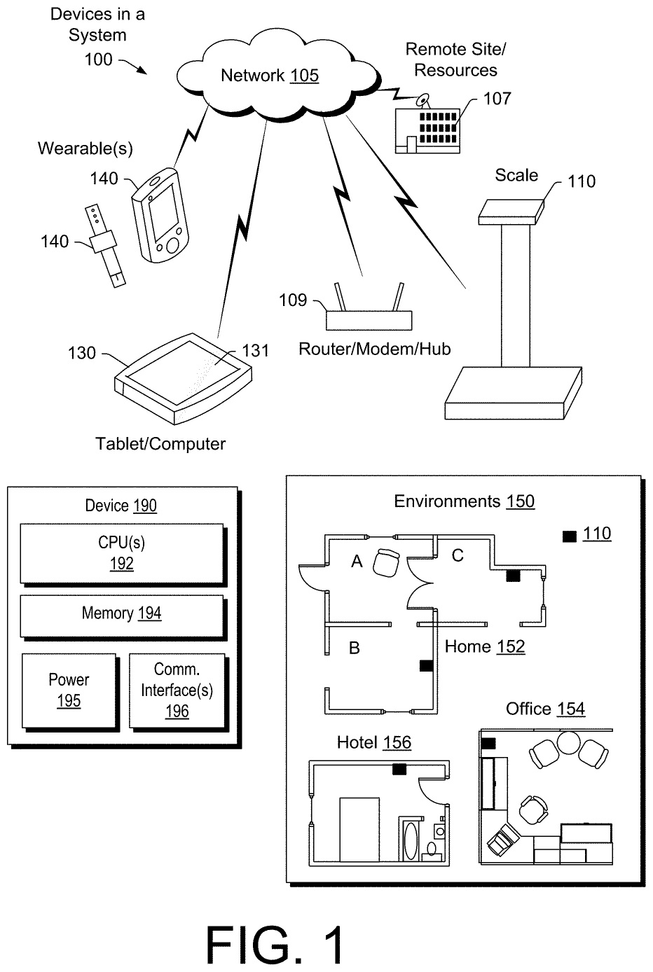

FIG. 1 shows an example of devices in a system 100 that includes one or more networks 105, one or more remote sites/resources 107, a router, modem and/or hub 109, a scale 110, a tablet/computer 130 with a display 131 and one or more wearables 140. As an example, one or more environments 150 can include one or more scales 110, for example, in a home 152, a hotel 156 and an office 154. As shown, a device 190 can include one or more processors 192, memory 194 accessible to at least one of the one or more processors 192, power 195 (e.g., a battery, a solar cell, a power outlet, etc.) and one or more interfaces 196.

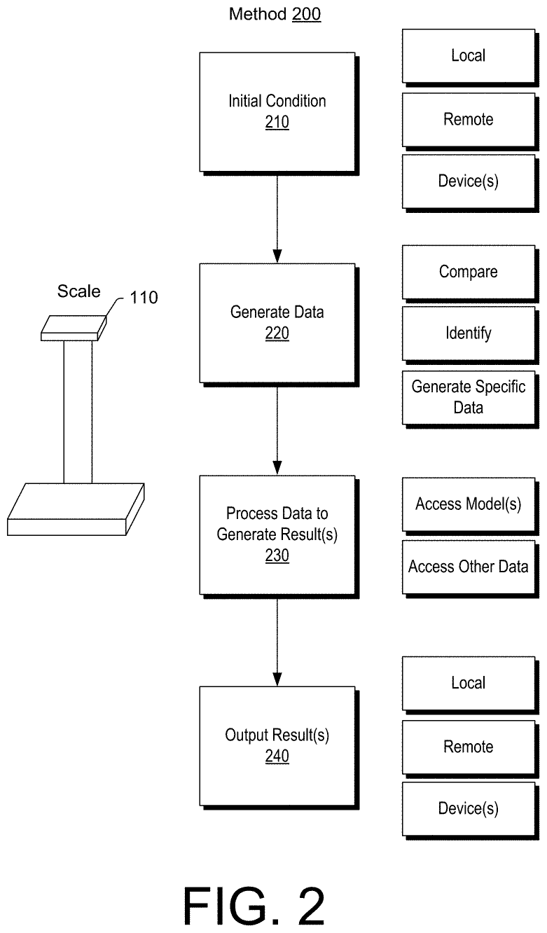

FIG. 2 shows an example of a method 200 that may be implemented, for example, with respect to the scale 110, which as a device, can include one or more features of the device 190 of FIG. 1. As shown, the method includes an initial condition block 210, a generation block 220 for generating data, a process block 230 for processing data to generate result(s), and an output block 240 for outputting result(s). In the example of FIG. 2, the initial condition block 210 may operate using data from one or more devices, systems, etc., which can include one or more of local, remote and devices (see, e.g., FIG. 1). As shown, the generation block 220 may include comparing, identifying and/or generating specific data. As shown, the process block 230 may include accessing one or more models and/or accessing one or more data stores. As indicated, the output block 240 can include outputting one or more results locally to local equipment, remotely to remote equipment and/or to one or more devices that may include the scale 110 and/or be other than the scale 110.

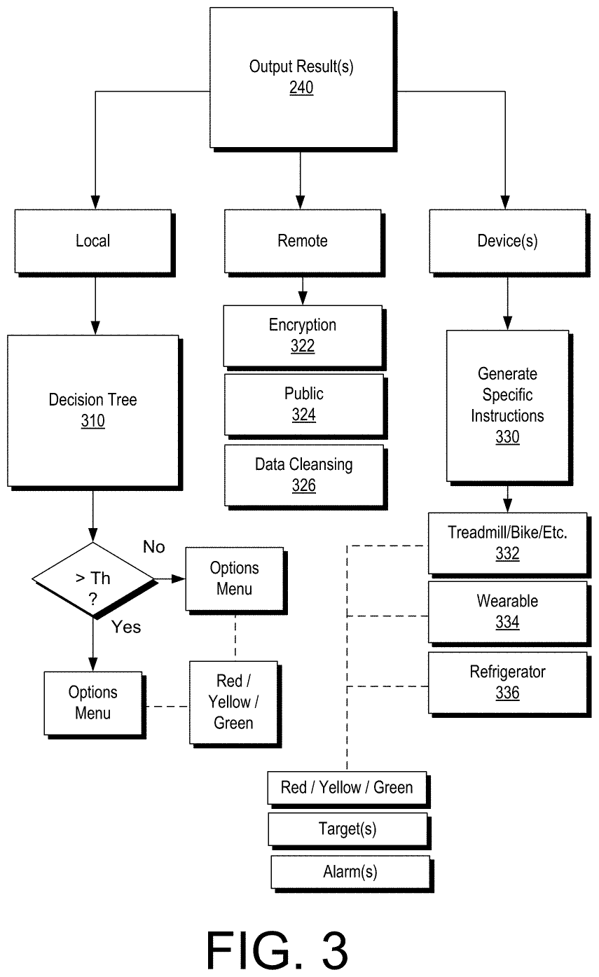

FIG. 3 shows an example of the output block 240 with respect to local, remote and device(s). As shown, a decision tree 310 can be implemented for purposes of making local output decisions, for example, as output to the scale 110 or, for example, one or more devices. A decision process is shown as an example where a threshold Th is utilized as a parameter to decide what options menu is to be rendered to a display and/or rendered audibly. An option may be via red, yellow and green lights (e.g., rendered graphics, etc.), which can convey an understandable indicator optionally without additional information, which could be subject to misinterpretation (e.g., overly negative or overly positive) and/or subject to de-motivation (e.g., as not meeting target goal(s), etc.). As an example, output may be controllable according to one or more settings.

As to the remote scenario, such an approach can include one or more of encryption 322, public availability 324 and data cleansing 326. For example, a user of the scale 110 may wish to have data private and protected via encryption and/or may wish to have data public. As an example, depending on service or services, some or all data may be subjected to cleansing, which may strip away particular data, etc. (e.g., as to identity, specifics, etc.).

As to the device scenario, a generation block 330 may generate specific instructions for one or more devices such as a treadmill, bike, etc. 332, a wearable 334 and/or an appliance such as a refrigerator 336 (e.g., a refrigerator freezer). As an example, where a user tends to order food via phone and/or call a personal instructor, the specific instructions may be for a phone (e.g., or text or messaging service). In such an example, consider texting a personal instructor to push the user harder to burn more calories, texting a pizza delivery service to not deliver particular pizzas to the user (e.g., possibly sending coupons for a selected number of health pizzas, etc.) or, for example, consider a talking refrigerator that can issue a message once the door is opened. As indicated, instructions can include, for example, red/yellow/green indicators, target(s), alarm(s), etc. For example, a wearable may be instructed to set an alarm that is for stopping activity where the alarm will not be triggered until an instructed level of activity is reached.

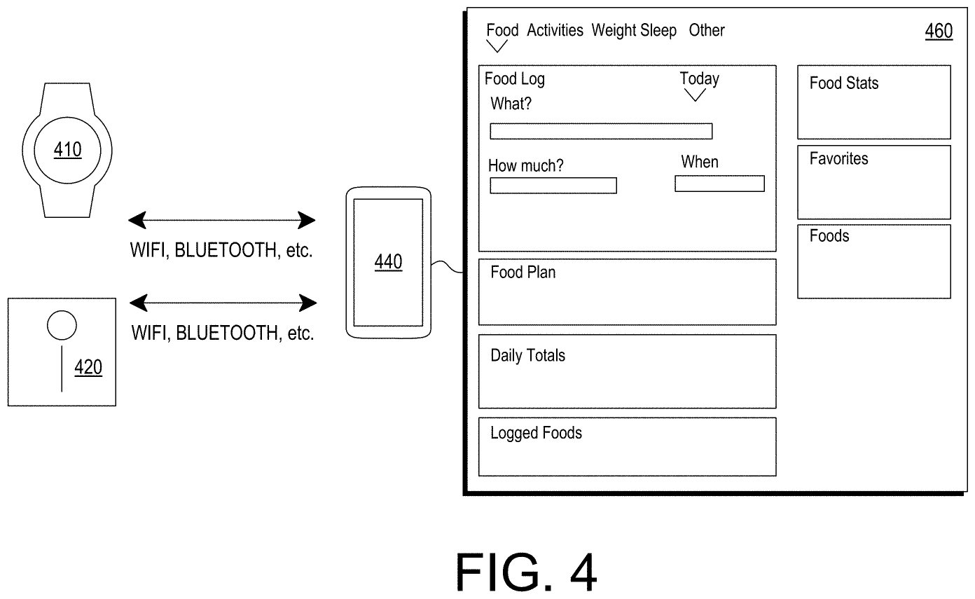

FIG. 4 shows an example of a system with various devices, including a wearable 410, a scale 420, a mobile device 440 such as a smart phone and a remote service 460 that can be an app that is operable at least in part via the mobile device 440. In the example of FIG. 4, the wearable 410 and/or the scale 420 may be in communication with the mobile device 440 and, hence, indirectly with the remote service. As explained with respect to FIG. 1, a device may be in more direct contact with a remote service. In the example of FIG. 4, the remote service 460 is shown as a graphical user interface (GUI), which can include, for example, various options accessible via one or more menus, graphical controls, etc. (e.g., food, activities, weight, sleep, other). As an example, one or more of the devices 410, 420 and 440 may acquire data via sensors, a network or networks and/or user input. For example, weight may be acquired via the scale 420, activity may be acquired via the wearable 410 and diet may be acquired via the mobile device 440 (e.g., via loT, user input, network, etc.). As an example, the remote service 460 may be operatively coupled to one or more supply sources of food and/or one or more healthcare providers, trainers, caretakers, etc.

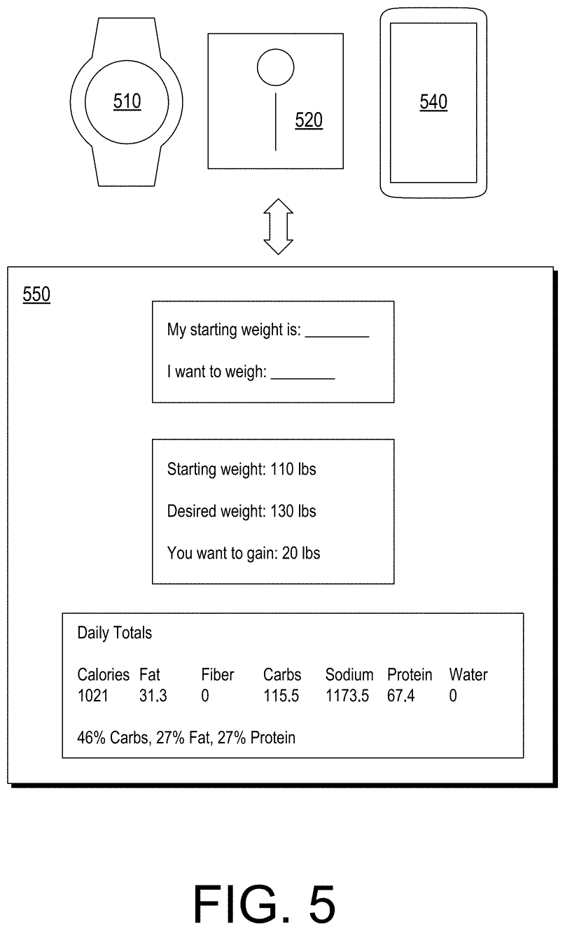

FIG. 5 shows various examples of devices 510, 520 and 540 along with graphical user interfaces (GUIs) 550 as rendered to a display of a device (e.g., pertaining to the FITBIT wearable app, a modified app, a different app, etc.). As to a wearable, consider the FITBIT wearable that tracks physical activity and can assist with diet using specific features that include recording calorie intake and meal quality to generate a fitness journal. The FITBIT system has a screen for fill in stats and intensity, enter current weight and the weight that to achieve; pick intensity of a food plan to create; FITBIT system outputs timing of achieving weight goal based on weight stats and intensity you chosen, from: Easier, Medium, Kinda Hard, and Harder. A process can then include click on the gear icon on a “Calories In vs. Out” tile (e.g., a spoon and fork icon). Then click the pencil icon (next to “Plan Intensity”). A final step of such a process displays the summary of the food plan. It includes current and desired weight, intensity selected, how many calories one will lose per day based on the intensity chosen. A process can include click the “Next” button again to finalize. The FITBIT system has a “Recording the Food You Eat” feature. For example, set the date and the Calories In vs. Out tile (spoon and fork icon) will appear on the page. A process can enter food intake details and click on Log, and then enter food. Foods can be entered manually or via scan of barcodes in the FITBIT app (tap the barcode icon and hold the barcode in front of your camera until the app says “Got it”). A user can fill out three details required for the food eaten: What did you eat? Type in the name of the food ate on the text field and a list of foods will appear. Select the most closely related item on the list, and it will populate in the text field. How much? Set the amount of the food ate by typing in the serving size you had on this text field. When? Click this drop-down list, and choose the time of the day that you had the food. The FITBIT system also has a feature to create meals to save time. In the FITBIT system, to monitor, enter food after every meal and snacks count too. Track diet, go to the “Food Log” section and click the “Log” button at the top of the dashboard to go to the Log page of the dashboard. Select the “Food” tab from the Log page to view diet records: the food plan then shows the number of calories that one can still eat that day to stay on track (also shows a view of calories consumed); and Logged Foods, which includes the food recorded in the food plan, here one can identify which foods can eat more and which one(s) may have to reduce. A process can include click the Calories In vs. Calories Out meter at any time to find out how close to plan's recommendation; where it will adjust as walk or exercise and as add meals and snacks to the food plan. Weight loss/diet guidance for humans is expanding as humans struggle to meet health and other goals. Different diets offer different solutions to obesity problems and/or fitness activities. Many diets focus solely on total weight loss, which is not necessarily a measure of health as there can be unhealthy means to achieve weight loss and a target weight.

The human body—as well as the body of every other animal—is mainly composed of four molecular-level components: water, fat, proteins and minerals, usually in that order of decreasing amounts. The substance that has attracted the most attention, from laypeople to medical professionals, is fat. This is, of course, motivated by the well-established fact that an excessive amount of body fat is related to increased morbidity and mortality. But also because adipose tissue (AT) is, by far, the most varying compartment— between individuals, but also within an individual over time. A widely used way to estimate body fat is the body mass index (BMI)—body weight normalized by height squared (kg/m²). Being a very simple and inexpensive method, it is the basis for WHO's definition of overweight (25 BMI<30) and obesity (BMI 30). However, for a given BMI, the body fat percentage changes with age, and the rate of this change is different depending on sex, ethnicity and individual differences. And while BMI correlates with fat accumulation and metabolic health in large populations, it is insensitive to the actual distribution of body fat.

When comparing methods for body composition analysis, a method may aim to distinguish fat (triglyceride) from AT, which contains approximately 80 percent fat, the rest being water, protein and minerals. While most of the body fat is stored in AT, fat is also present in organs such as liver and skeletal muscle. The metabolic risk related to fat accumulation is strongly dependent on its distribution. Central obesity and, in particular, ectopic fat accumulation are important metabolic risk factors. Large amounts of visceral AT (VAT) are related to increased risks of health issues such as cardiac risk, type 2 diabetes, liver disease and cancer. High levels of liver fat increase the risk for liver disease and type 2 diabetes, and increased muscle fat has been associated with increased risk for insulin resistance and type 2 diabetes 16 and reduced mobility. While there are other anthropometric measures, such as waist circumference and waist-to-hip ratio, which more strongly correlate with metabolic risk, BMI and various other anthropometric surrogate measures may be suboptimal predictors for individual fat distribution and metabolic risk.

Besides fat, acting as the body's long-term energy storage, skeletal muscles are of great interest to study, and the balance between the energy-consuming muscles and the energy-storing fat compartments is, of course, highly relevant in order to understand the metabolic balance of the body. Cachexia, involuntary loss of body weight, usually with disproportionate muscle wasting, is a life-threatening condition, often related to the progression of an underlying serious disease (e.g., cancer). In cancer, cachexia is defined as weight loss of >5 percent over 6 months, BMI<20 kg/m² or appendicular muscle mass normalized by body height squared of <7.26 kg/m² or 5.45 kg/m² for males and females, respectively. Sarcopenia, which can be related to cachexia, but is also associated with aging, is often defined as reduced physical performance following loss of muscle mass, usually accompanied by increased fat infiltration of the muscles. When diagnosing sarcopenia, muscle strength tests combined with muscle volume measurements are helpful. Muscle pathology progression over 1 year could be detected by quantitative MRI but not by assessing muscle strength or function. These examples illustrate a demand for more sophisticated body composition analysis tools.

As explained, when it comes to weight loss, an aim may be to lose as much fat tissue as practical, while retaining as much muscle tissue as possible, or for some it may be better: to increase the muscle mass, while simultaneously decrease the mass of fat, in the human body. As an example, a minimum target may be set to a healthy target level, for example, between approximately 6 percent to approximately 8 percent (e.g., in males). A minimum level may aim to provide for health, such as lubrication, protecting intestines against shock, thermal insulation, etc. In some instances, risk of injury can be increased where level of fat falls below a certain level.

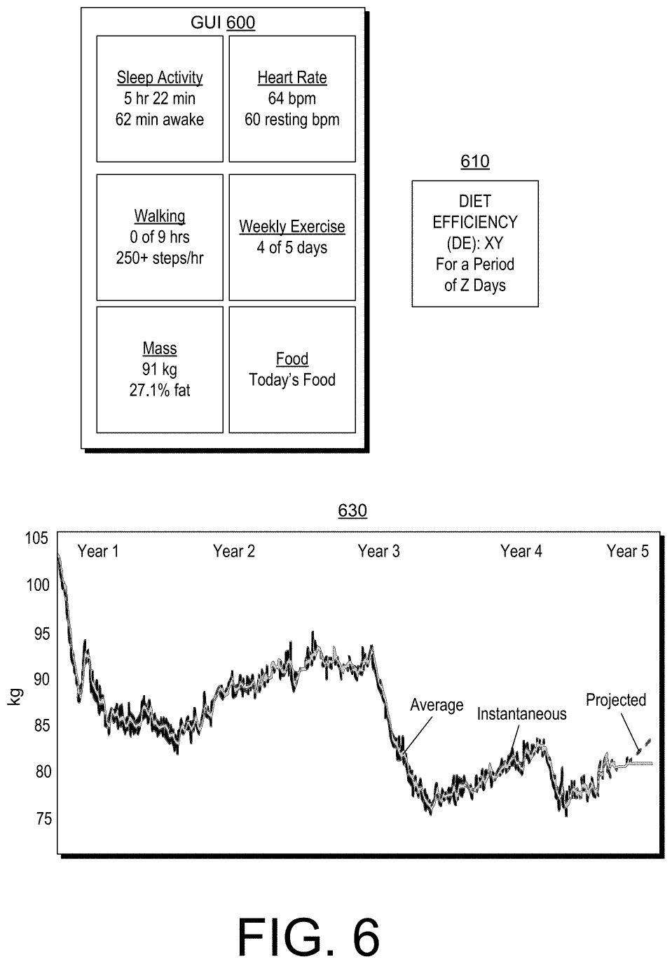

As an example, a desirable objective can be numerically quantified, for example, by a parameter referred to as “Diet Efficiency” (DE): Diet Efficiency=100*ratio of fat tissue loss (gain)/ratio of total tissue loss (gain). The unit of Diet Efficiency is (%). Such a parameter may be, for example, referred to as “Fat Burn Ratio”, “Diet Performance”, “fitness performance”, etc. The DE measure depends on two reliable measurements: 1) the total change in body tissue mass (ΔW, total weight change) and 2) the change in fat tissue mass (ΔW_{f}), over a given timespan (ΔT). As an example, a DE measure may be computed using circuitry of a scale, which can provide body tissue mass readings. Such a scale may be operatively coupled (e.g., via wire and/or wirelessly) to one or more other devices. As an example, such a scale may include a display that can render one or more graphical user interfaces (GUIs) for input and/or output. As an example, a scale may include a camera, a barcode reader, an IRFD reader, etc. As an example, a scale may receive and/or acquire information sufficient to compute DE and, for example, to render output (see, e.g., the GUIs of FIGS. 6 to 11, etc.).

As an example, a workflow can be performed to quantify a DE measurement for a human. As an example, a set of utility tools can be implemented realize the workflow in working equipment. As an example, instructions may be executed using circuitry of a device or a system that can explain how one can utilize tools, modify behavior, reward behavior, punish behavior, provide recommendations of services and/or tools, provide access to services and/or tools, etc.

As explained, an exact mean to quantify the quantity ΔW_{f} can involve one or more scans of a body, for example, using expensive professional multi-physics equipment, which at a minimum may be via a weight scale and electrical resistance analysis (ERA), while more advanced techniques involve MRI, CT, ultrasound, etc.

As to a weight scale and ERA, consider an article by Miller et al., (October 2016), entitled: “Validating InBody® 570 Multi-frequency Bioelectrical Impedance Analyzer versus DXA for Body Fat Percentage Analysis”, Journal of Exercise Physiology online, 19: 71-78. ISSN 1097-9751, which is incorporated by reference herein. The InBody 570 equipment generates a Result Sheet, where each InBody Test will print out a full-page detailing the muscle, fat, and water values of the user. The InBody 570 provides lean mass and fat values in each segment of the body to give an assessment of body composition. It provides for segmental fat and lean mass analysis, which can be used to identify how many pounds of lean mass are in each body segment, which may be used to determine how specific diets and exercises are affecting the body composition. It also provide for injury identification, which can be for identification and tracking of inflammation, swelling and joint injuries with ECW/TBW Analysis.

There are also low-end, relatively inexpensive equipment such as weight scales, which may offer various measurements, though they tend to be less accurate and trusted as a gross indicator through repeat measurements averaged over many days. As an example, consider the FITBIT ARIA 2 “smart scale” that aims to track weight, body fat percentage, BMI (body mass index) and lean mass.

As current tools tend to be expensive or inaccurate, it can be desirable to take an approach that provides quick and accurate feedback of diet performance. Rapid feedback can be beneficial for morale (positive results may provide moral support to dieters) and for body composition (as it can be unfortunate to lose muscle tissue, as it is time-consuming, exhaustive and expensive (need for quality food) to recover the lost muscle mass). Such an approach may be a stand-alone approach and/or an integrated approach, where it is integrated into one or more pieces of equipment, one or more services, etc.

As mentioned, a method can include calculating an accurate estimate of DE, which can be based on a limited set of measurements. For example, consider an approach that relies on two measurements:
Food Energy Deficiency (ΔE) over a desired time interval. The unit used in this work is calories (kcal); and
Weight Change (ΔW) over approximately the same time desired interval. Such a measurement may be via a weight scale (e.g., a consumer-goods type of weight scale, an instrumented weight scale, a “smart” weight scale, etc.).

As an example, Energy Deficiency (Delta Energy: ΔE) can be calculated by two measurements: ΔE=Energy Gained—Energy spent (over unit time). Energy gained is the amount of energy intake (eaten) over the unit time, and the energy spent is the amount of energy the body has spent over approximately the same time span.

As an example, such a measurement may be approximated by an activity/fitness trackers (e.g. FITBIT, JAWBONE, smartphone with built-in activity meters, etc.), or using a formula. As to a formula, consider the Mifflin-St Jeor Equation.

As an example, a Calorie Calculator may be based on several equations, and the results of the calculator based on an estimated average. The Harris-Benedict Equation may be used to calculate basal metabolic rate (BMR), which is the amount of energy expended per day at rest. It was revised in 1984 to be more accurate when the Mifflin-St Jeor Equation was introduced. The Mifflin-St Jeor Equation also calculates BMR, and has been shown to be more accurate than the revised Harris-Benedict Equation. The Katch-McArdle Formula is slightly different in that it calculates resting daily energy expenditure (RDEE), which takes lean body mass into account, something that neither the Mifflin-St Jeor nor the Harris-Benedict Equation do. Of these equations, the Mifflin-St Jeor Equation is considered to be relatively accurate for calculating BMR with the exception that the Katch-McArdle Formula can be more accurate for people who are leaner and know their body fat percentage. Mifflin-St Jeor Equation is for men: BMR=10W+6.25H−5A+5 and for women: BMR=10W+6.25H−5A-161. The Revised Harris-Benedict Equation is for men: BMR=13.397W+4.799H−5.677A+88.362 and for women: BMR=9.247W+3.098H−4.330A+447.593. The Katch-McArdle Formula is BMR=370+21.6(1−F)W, where W is body weight in kg, H is body height in cm, A is age, and F is body fat in percentage.

A value obtained from one of the foregoing calorie calculator equations is the estimated number of calories a person can consume in a day to maintain their body-weight, assuming they remain at rest. This value may be multiplied by an activity factor (e.g., 1.2-1.95), dependent on a person's typical levels of exercise, in order to obtain a more realistic value for maintaining body-weight (since people are less likely to be at rest throughout the course of an entire day). 1 pound, or approximately 0.45 kg, equates to about 3,500 calories. As such, in order to lose 1 pound per week, it can be recommended that 500 calories be shaved off the estimate of calories necessary for weight maintenance per day. For example, if a person has an estimated allotment of 2,500 calories per day to maintain body-weight, consuming 2,000 calories per day for one week would theoretically result in 3,500 calories (or 1 pound) lost during the period.

Proper diet and exercise is largely accepted as a way to lose weight. It tends to be inadvisable to lower calorie intake by more than 1,000 calories per day, as losing more than 2 pounds per week can be unhealthy, and can result in the opposite effect in the near future by reducing metabolism. Losing more than 2 pounds a week may likely involve muscle loss, which in turn lowers BMR, since more muscle mass results in higher BMR. Excessive weight loss can also be due to dehydration, which is unhealthy. Furthermore, particularly when exercising in conjunction with dieting, maintaining a good diet can be beneficial as the body needs to be able to support its metabolic processes and replenish itself. Depriving the body of the nutrients it requires as part of heavily unhealthy diets can have serious detrimental effects, and weight lost in this manner has been shown in some studies to be unsustainable, since the weight is often regained in the form of fat (putting the participant in a worse state than when beginning the diet). As such, in addition to monitoring calorie intake, it can be beneficial to maintain levels of fiber intake as well as other nutritional necessities to balance the needs of the body.

A tedious number to quantify is the amount of energy consumed (e.g., eaten and/or drunk). To do so, a person may need calorie tables and food weight numbers. This number however may be readily approximated with useable digital tools, like a mobile app, or a customizable web service. One can, for example, exploit the fact that humans are creatures of habit where, for example, one tends to have the same breakfast every single working day (and similar for weekend meals). One may also exploit that dieters are often following a diet plan with pre-calculated calorie counts (provided e.g. by Personal Trainers or recipes found in books/online). It is now also coming Al-based products on the market for this purpose. One Al example is a mobile app where the user can take a photo of every consumed item, and the app will proceed to estimate the energy content of the photographed items, and log them in a personal diary.

As explained, as an example, a method can estimate a DE value using the numbers above (energy deficiency and weight deficiency), with no extra information about the composition (proteins, carbohydrates or fat) of the consumed food, or the activity level/fitness regime of the person.

An article by Hall, entitled “What is the required energy deficit per unit weight loss?”, Int J Obes (Lond). 2008; 32(3):573-576. doi:10.1038/sj.ijo.0803720, which is incorporated by reference herein, states that an estimate for weight loss is that a cumulative energy deficit of 3500 kcal is required to lose 1 pound of body weight, or equivalently 32.2 MJ per kg. Such a rule assumes exclusive loss of adipose tissue consisting of 87% fat. When energy intake does not meet energy requirements, the deficit may be accounted for by metabolism of stored energy in the form of body fat, protein, and glycogen. As energy is conserved, metabolizable energy content of lost tissue can be equivalent to an energy deficit to produce that weight loss. The metabolizable energy density of the lost tissue may be determined by its chemical composition. Loss of body water may result in a mass change but it may not contribute to metabolizable energy content. Metabolizable energy densities of body glycogen, protein and fat are approximately 17.6, 19.7, and 39.5 MJ/kg, respectively.

As to an underlying basis of an example of a DE approach, consider the water fractions of fat tissue and muscle tissue. As to water in fat tissue, consider an article by DiGirolamo et al., entitle “Water content of rat adipose tissue and isolated adipocytes in relation to cell size”, Am J Physiol. 1976 November; 231 (5 Pt. 1):1568-72, which is incorporated by reference herein. DiGirolamo et al. studied epididymal adipose tissue composition and adipocyte water content in male rats during growth and development of spontaneous obesity. The data show that a highly significant positive correlation exists between fat-cell volume and intracellular water space (IWS) (r=0.967, P less than 0.001). Intracellular water, expressed as picoliters per fat cell, varied from 1.5-2 in small fat cells (mean vol, 30-50 pl) to 9-10 in large cells (800-1,000 pl). When expressed as percent of fat-cell volume, IWS varied from 5-7% in the small fat cells to 1-1.3% in the large ones. Total adipose tissue water continued to increase with increasing adipose mass. Similarly, total adipocyte water increased with enlarging cell size and tissue mass. The contribution of total adipocyte water (as contrasted to that of nonadipocyte water) to total tissue water, however, was found to be limited (less than 23%) and to decline progressively with adipose mass expansion.

An article by Wang et al., entitled “Disparate hydration of adipose and lean tissue require a new model for body water distribution in man”, J Nutr. 1976 December; 106(12):1687-93, which is incorporated by reference herein, studied the intra and extracellular fluid spaces in adipose tissue by 3H₂O, 82Br-(minus), and 35SO4=uptake, and by appropriate chemical methods, in surgical biopsy material from 16 patients undergoing elective laparatomy. Total water space for adipose tissue was 14+/−1.4%: extracellular component was 11+/−1.1% in mesenteric and subcutaneous depots. Use of these adipose tissue hydration constants, combined with measurements of total body water (TBW), extracellular water (ECW), total body potassium, and experimentally derived age-specific constants for lean body potassium content, permits development of a four compartment model for body water which considers intra and extracellular components separately for adipose (AT) and adipose-free (AFM) tissue masses. This model has a form where x is the total hydration and y the extracellular hydration of the adipose-free mass. The equations can be solved for the ECW and ICW of the adipose-free mass, defined by its potassium content. In four normal subjects, x was measured as 0.80+/−0.032, and y as 0.24+/−0.017. Thus, the adipose free mass, compared with adipose tissue, has approximately six times the total water and twice the extracellular water content per unit weight.

For humans, in various examples herein, muscle tissue can be approximately 25% protein and 75% water, while fat tissue can be approximately 90% fat and approximately 10% water and blood. The numbers for fat tissue can be compared with DiGirolamo et al. for rat AT, where intercellular water space (IWS) as a percent of fat-cell volume varied from 5-7% in the small fat cells to 1-1.3% in the large ones, and can be compared to Wang et al., where “adipose tissue hydration constants” are from determinations of total water space for adipose tissue being 14+/−1.4% where an extracellular component was 11+/−1.1% in mesenteric and subcutaneous depots.

Below, examples of series of equations are presented for loss of fat tissue calculations, determinations, etc. One or more of such equations may be computerized, for example, as a program that is local and/or remote or as programs that may be local and/or remote.

Loss of fat tissue may be quantified as follows: ΔW_{f}+ΔWₘ=ΔW, where ΔW is ending velocity factor (g), ΔW_{f} is ending velocity factor fat and ΔWₘ is ending velocity factor muscle.

As to energy, consider the following balance: ΔE_{f}+ΔEₘ=ΔE, where ΔE is ending energy, ΔE_{f} is ending energy fat and ΔEₘ is ending energy muscle.

Further, consider eₘ is energy density protein, of is energy density fat, swₘ is water content muscle, and sw_{f} is water content fat such that:

ΔE_{f}=ΔW_{f}(1−sw_{f})*e_{f}) and ΔEₘ=ΔWₘ(1−swₘ)*eₘ)

ΔW_{f}=(ΔE−ΔW(1−swₘ)eₘ)/((1−sw_{f})e_{f}−(1−swₘ)eₘ)

Consider, for example, the following values: swₘ=0.75, eₘ=4 kcal/g and sw_{f}=0.10, of =9 kcal/g. As to derivation of an equation using the foregoing values, consider:

ΔW_{f}=(ΔE−ΔW(1−0.75)*4)/((1−0.1)*9−(1−0.75)*4)

ΔW_{f}=(ΔE−ΔW)/(0.9*9−1)

ΔW_{f}=(ΔE−ΔW)/7.1[units g]

Example A: ΔE=2000 kcal, ΔW=300 g, ΔW_{f}=(2000−300)/7.1=1700/7.1 and ΔW_{f}=239 g fat tissue. Example B: ΔE=2000 kcal, ΔW=200 g, ΔW_{f}=(2000−200)/7.1=1800/7.1, and ΔW_{f}=253 g fat tissue. Example C: ΔE=2000 kcal, ΔW=0 g, ΔW_{f}=(2000)/7.1−(1/7.1)*ΔW, ΔW_{f}=282−0.14*ΔW, and ΔW_{f}=253 g fat tissue.

As explained, the mass of lost fat and the mass of lost muscles can be closely approximated to be: ΔW_{f}=(ΔE−ΔW)/7.1 and ΔWₘ+ΔW−ΔW_{f} and DE=100*ratio of fat tissue loss (gain)/ratio of total tissue loss (gain)=100*(ΔW_{f}/ΔW).

Where ΔE is the difference between consumed energy (through food and drinks) and consumed energy, over a given timespan, and the unit is kcal. ΔW is the total change in body weight (measured in gram unit) over the same time-span. ΔW_{f} is the part of that total weight change ΔW, which is due to loss (gain) of fat tissue. ΔWₘ is the corresponding change (loss or gain) of muscle tissue. Corresponding equations for any other unit of measurement (other than gram, kcal) may be readily derived (e.g., via a conversion algorithm, etc.).

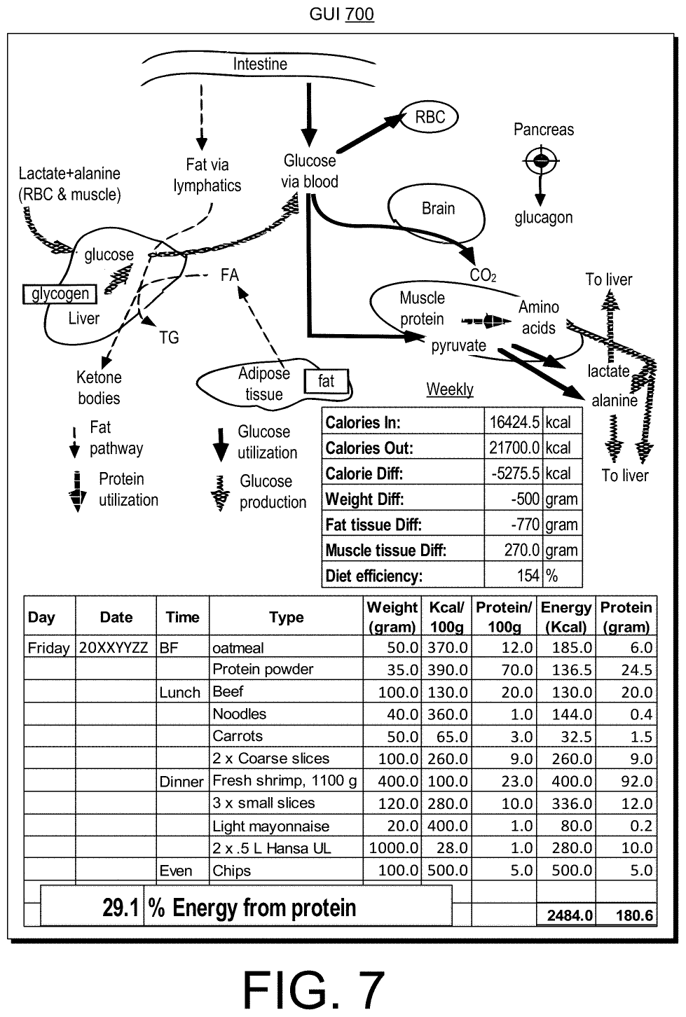

The effect of this formula can be exemplified through the spreadsheets of FIGS. 6 and 7, for daily measurements (NB: using the term “Fat Burn Fraction” instead of DE in these two examples), for an example user-specified energy deficiency ΔE of −1500 kcal/day, and, as measured over a one-week timespan, for an example energy deficiency ΔE of −7000 kcal/week.

As an example, if a person has a weekly energy deficiency of 7000 kcal, and looses 1000 gram of weight over the same time-span, then one can determine that the person will have lost 845 grams of fat tissue and 155 grams of muscle tissue. This is then equivalent to a DE (or “Fat Burn Rate”) of 85%, which may be considered to be a “healthy” weight loss (e.g., because the person loses much more fat mass than muscle mass). On the other hand, if the person observes a weight loss of 2000 gram for the same energy deficiency, then the person will have lost 704 grams of fat tissue and 1296 grams of muscle tissue, which is sub-optimal as the person then will have lost almost twice as much muscle tissue than fat tissue (e.g., essentially has lost mainly water, since muscle tissue is approximately 25% protein and 75% water, while fat tissue contain approximately 90% fat and 10% water and blood). Also, if a DE value is greater then 100% then the person has lost more fat tissue than the total weight change, which can happen if the person at the same time has gained muscle tissue. Such a scenario may be optimal or ideal, which may be referred to as a “super-dieting” state: loose fat and gain muscle at the same time. External conditions where this state is achieved may be quite complicated, and may consist of an optimal combination of protein-rich food and an efficient exercise regime.

The derivation of the approximate equation for DE can include one or more assumptions. An exact equation for ΔW_{f} can be: ΔW_{f}=(ΔE−ΔW(1−swₘ)eₘ)/((1−sw_{f})e_{f}−(1−swₘ)eₘ).

An assumption can be constant values for swₘ (water content/saturation of muscle tissue=75%), sw_{f} (water content/saturation of fat tissue=10%), eₘ (calories per gram of protein=4 kcal) and of (calories per gram of fat=9 kcal), in order to get to the approximate equation: ΔW_{f}=(ΔE−ΔW)/7.1.

The foregoing values may vary somewhat between individuals (in particular the water saturations can vary between sexes and ages). An approach may involve using customized values, which may be constants (see, e.g., exact formula). As to an approach for qualitative results that can be readily understood via graphics, short words, etc., (good, bad, fair, excellent, etc.), the approximate formula may be adequate. As to uncertainty of measurements, in particular the consumed energy part, the uncertainty in the saturation properties may be of a second order of impact.

As an example, a method, device, assembly, system, etc., may utilized and/or may include one or more types of machinery (e.g., a spreadsheet, computer program, personal activity tracker, mobile phone app, smart watch, standalone “smart” weight scale, etc.), which may allow a user to manually and/or automatically enter (e.g., estimated and/or measured) measurement quantities of a) weight change, b) activity level (measured in e.g. calories per unit time) and c) consumed energy in a given period (measured e.g. in calories). Based on these three inputs, as an example, machinery may output an estimate of DE over the measured time-span. Such an approach may be via one of the two equations, or approximations of those two equations, specified above.

As to an example implementation, consider an activity-tracker, a weight scale, a cell phone, and an (internal or external) application/utility which tracks (or by other means estimates) the consumed energy for a person over time (see, e.g., FIG. 4). In such an implementation, information can be computed in an app (executing in the central compute device) which integrates all information, and computes DE. As an example, an implementation may be via a healthcare app, which may include a graphical user interface (GUI).

FIG. 6 shows an example of a GUI 600 that can include one or more graphical components where, for example, one of the graphical components can render information for DE 610 and where, for example, one of the graphical components can render one or more plots 630. For example, DE could be provided in a separate “tile” in this application, where weight, diet details and activity level are already logged and available, either in standalone mode, or through a web service. The plot 630 shows historical data of mass versus time with average (7 day) and instantaneous and projected (e.g., computed). As an example, such a plot may include information as to DE, hydration, BMI, etc.

Various components of a weight loss framework (e.g., a computational framework) may include a DE feature. As an example, in the absence of an activity tracker, equipment may use an alternative empirical relationship to calculate amount of spent energy (for example, based on the well-known Mifflin-St Jeor Equation).

As an example, an approach may provide an option to “get away” without an explicit food/beverage logging application and instead use a pre-calculated calorie(s) estimate(s) provided from a table or tables (e.g., possibly by specifying a users weight, age, sex and lifestyle choices).

As an example, a weight loss framework can include and/or be operatively coupled to a device to measure change in weight over time; noting that absolute weight is not necessarily demanded and that a measurement and/or estimate of weight change over time may suffice. In such an example, a minimum configuration of an equipment system can involve a weight scale and circuitry to compute diet efficiency (DE), for example, as based on estimates of activity level and energy intake.

As an example, a “smart” weight scale can include one or more types of circuitry that can provide for output of DE. For example, a smart weight scale can be configured where information other than weight can be entered/estimated through additional information (possibly pre-provided, like sex, age, activity level, diet profile) provided by a user (e.g., via a user interface, with keys, touchscreen, voice commands, etc.). Such a scale may then provide an estimate of DE. As an example, a smart scales may include a network interface or network interfaces (e.g., BLUETOOTH, WIFI, etc.). As an example, a user interface may be provided through an “app” on a secondary compute device (e.g. a remote control, a mobile phone, a smart refrigerator, etc.).

As an example, if weight change can be estimated by other means (e.g. manual readings from “dumb” weight scales, “gut feeling”, empirical tables, etc.), then an approach may be implemented via a computing device that includes circuitry for implementing the calculation of DE. As an example, consider a smart phone, a smart watch, a smart refrigerator, a smart food scale (e.g., for weighing portions of food, etc.).

As an example, a smart watch can include a built-in activity tracker. In such a device, DE may be calculated using such an app, if weight change is provided, either manually, automatically (e.g. through a BLUETOOTH or other connection to a measuring device) or as an empirical estimate of weight change.

In the very simpest use-cases/implementations of this invention, where DE is calculated based on one or more estimated input values, with higher uncertainty in input values than what we get with real physical measurements, it may be more useful to use some qualitative means to indicate level of DE than through a percent-estimate (which would give an unrealistic appearance of high accuracy of measurement).

As an example, a graphical user interface (GUI) can include a performance indicator for DE, where %-threshold values could be indicated by needle position (e.g., a gauge approach).

FIG. 7 shows an example of a GUI 700 that can include one or more biological pathways with associated organs, etc., along with various types of data, including inputs and outputs. As an example, the GUI 700 may facilitate understanding of metabolism related to DE, where, for example, various DE pathways may be highlighted to be indicative of a DE value and, for example, one or more possible DE values that may be targets. The tables of data can be for data on a particular basis such as daily, end of each week (e.g., a seven-day running average), etc. The data present a computed estimate of change in fat tissue and change in muscle tissue. Such data were confirmed using professional body scans where there was an acceptably good correlation between the computed estimates and the actual measurements. In particular, there was less than a 0.5% of fat change difference between measurement and estimate. As an example, hydration information may be included in a GUI. For example, consider a hydration as may be associated with muscle. As an example, a GUI may include rendering a hydration indicator that provides information instructive for increasing and/or decreasing intake of water, which may be wate that is not associated with protein at the time of intake (e.g., drinking water, intravenous water, etc.).

As shown, the pathways include adipose tissue and muscle. An article by El Bacha et al., Dynamic Adaptation of Nutrient Utilization in Humans, Nature Education 3(9):8 (2010) is incorporated by reference herein. Various types of pathways may be highlighted using one or more graphical user interfaces. As an example, an animal may burn protein to create some amount of fat and/or some amount of new protein. As an example, a graphical user interface can be utilized in combination with one or more pathways where such a GUI may be part of a social application, an exercise application, a scale application, etc. As an example, badges, etc., may be indicated in a “game” type of environment where certain pathways can be associated with particular badges.

As an example, aspects of the brain-gut axis may be included in determining, adjusting, etc., a diet. For example, consider a mood/psychology aspect to diet. In such an example, sugars, carbohydrates, etc., may be limited or otherwise controlled.

In general, the gut-brain axis (or brain-gut axis), is a bidirectional neurohumoral communication system, that is involved in maintaining homeostasis and is regulated through the central and enteric nervous systems and the neural, endocrine, immune, and metabolic pathways, and especially including the hypothalamic-pituitary-adrenal axis (HPA axis).

As an example, gut flora can produce a range of neuroactive molecules, such as acetylcholine, catecholamines, γ-aminobutyric acid, histamine, melatonin, and serotonin, which are essential for regulating peristalsis and sensation in the gut. Changes in the composition of the gut flora due to diet, drugs, or disease correlate with changes in levels of circulating cytokines, some of which can affect brain function. The gut flora also release molecules that can directly activate the vagus nerve, which transmits information about the state of the intestines to the brain.

Likewise, chronic or acutely stressful situations activate the hypothalamic-pituitary-adrenal axis, causing changes in the gut flora and intestinal epithelium, and possibly having systemic effects. Additionally, the cholinergic anti-inflammatory pathway, signaling through the vagus nerve, affects the gut epithelium and flora. Hunger and satiety are integrated in the brain, and the presence or absence of food in the gut and types of food present also affect the composition and activity of gut flora. As an example, one or more adjustments may be made on the basis of gut flora. For example, an individual may utilize one or more types of “biotics” where such organisms may impact digestion, absorption, metabolism, etc. In such an example, an amount of nutrients which enters the blood stream may be smaller than the amount of nutrients digested where, for example, the fraction entering the blood stream may be a function of gut flora. In such an example, a method may utilize a lower “effective” calorie constant for a nutrient at hand. For example, consider utilization of data (e.g., empirical experience, etc.) where 3.4 calories/gram for protein is utilized rather than 4.0 calories/gram for particular intake or intakes. As explained, energy density values may be constants and/or may be variables in various equations. Certain diseases may cause metabolic realities that differ from those of a normal individual (e.g., consider tumor cells, etc.).

As an example, a method, a device, an application, a system, etc., can provide feedback to a user as to encouraging a diet, which may consider eating of whole foods, nutrient-dense meals, snacks, etc. As an example, an approach can include providing a user with a questionnaire that helps to tailor a diet with aspects of a user's life, which can include user goals, medical conditions, etc. As an example, a method can include generating a personalized energy plan, which can include a diet with calories from one or more sources (e.g., protein, fat, and carbohydrates, etc.).

As an example, a method may have a user maintain a log as to diet, exercise, weight, mood, etc. Such a log may be in part via a GUI or GUIs and/or via scanning, photography, sensors (wearable, external, etc.), etc. As an example, a method may provide for opportunities for a user to update one or more goals, types of information, etc., which may be utilized to revise a diet.

As an example, an approach may implement additional machinery to provide predictive power. For example, there can be an approach for explanatory power (given the measurements, this is what's happened) and an approach for predictive power where the two may optionally be integrated (e.g., combined).

As an example, predictive power may predict that, for a given level of activity and consumption, a person will experience this or that weight loss and/or DE, based on specific information about the individual person seeking efficient diet regime. Such an approach may be implemented by providing (e.g., optionally building) a database that includes real-world observations of DE for a sufficient population of persons, containing a collection of personal attributes which may have influence over the real-world DE performance. As an example, a set of attributes for the real-world cases could include one or more of the following facts, for each entry in the database: Food intake, Weight loss, Activity level, Sex, Age, Weight, Height and Occupation.

Based on these attributes, it can be determined, over time, accurate statistical models over situation-based DE. Such an approach may be accomplished through regression-fitting (e.g., a least-squares method), unconstrained machine learning of a statistical model, etc.

As an example, an approach may “gamify” dieting activity. Such an approach can include creating an user community, where individuals can compare DE scores, rank their performance with peers (e.g. of same age, sex and weight range), and exchange diet and exercise details and advices.

As an example, an implementation can include integrating various features into a fitness tracker/management application. For example, consider an implementation that involves one or more of FITBIT, GARMIN POLARIS, APPLE watch, SAMSUNG GEAR, APPLE IPHONE, etc., equipment and/or services.

As an example, an implementation can be via one or more personal trainers, or another body providing diet/exercise/lifestyle services, which may provide customized services, where one will measure and optimise the DE individually for a portfolio of customers.

As an example, one or more methods, devices, systems, etc., can be utilized in animals, which can be other than humans. For example, consider one or more of the livestock, equine, domestic pets (e.g., dogs, cats, etc.), etc. As an example, an application may be utilized for diets of one or more types of animals. Such an application may aim to achieve an amount of fat, an amount of muscle, a weight, a ratio of fat to muscle, etc. As an example, one or more animal scales, animal wearables, etc., may be utilized for achieving one or more goals.

As example, where weight is a target, muscle may be desirable over fat to the extent that muscle takes less energy to develop than fat. As explained, one kilogram of fat is a substantial amount of energy, particularly when compared to one kilogram of muscle, which includes a substantial amount of water.

As an example, a method can include assessing parameters of animals that are to be fed a particular diet. Such parameters may include weight, fat, muscle, bone, organs, etc. As an example, a constant may be bone and organ mass where fat and muscle are variables.

An article by Albrecht et al., Growth- and breed-related changes of marbling characteristics in cattle, Journal of Animal Science, Volume 84, Issue 5, May 2006, Pages 1067-1075, https://doi.org/10.2527/2006.8451067x (1 May 2006), is incorporated by reference herein. The article by Albrecht et al. pertains to growth- and breed-related changes of marbling characteristics in cattle. Albrecht et al. studied four cattle breeds with different growth impetus and muscularity. German Angus, as a typical beef cattle; Galloway, as a smaller, environmentally resistant beef cattle; Holstein-Friesian, as a dairy-type cattle; and double-muscled Belgian Blue, as an extreme type for muscle growth, were used. These 4 breeds were expected to have differences in muscle development and i.m. fat deposition. Marbling characteristics were determined and classified in LM and semitendinosus muscle by computerized image analysis. Among breeds, differences appeared in the quantity, structure, and distribution of the marbling flecks in both muscles. The deposition of fat in the double-muscled Belgian Blue bulls remained substantially inferior to that of the other breeds, up to the age of 24 months (mo). Marbling in German Angus bulls particularly showed larger (P<0.05) marbling fleck areas. Galloway cattle had the greatest (P<0.05) number and the most regular (P<0.05) distribution of the marbling flecks in young animals. Furthermore, for marbling characteristics in Holstein-Friesian animals, a great number and slightly finer structure were observed compared with the other breeds investigated. Postnatal growth-related changes of marbling in LM were characterized by as much as a 40-fold increase in the number of marbling flecks from 2 to 24 mo of age but also by up to a 4-fold enlargement in the area of the marbling flecks. The structure of marbling flecks was determined by 2 development trends. On the one hand, the marbling flecks became larger (P<0.05), and the structure became coarser, which was reflected by an increasing (P<0.01) proportion of long marbling flecks as well as an increasing (P<0.01) maximum skeleton line length. On the other hand, continually new small, round marbling flecks appeared. This caused a decrease (P<0.01) in the proportion of the 3 largest marbling fleck areas. The distribution of the marbling flecks became more regular (P<0.05) with increasing proportion and number of marbling flecks. The results suggest that hyperplasia of adipocytes plays an important role in marbling during growth of muscle in cattle.

As an example, a method can include targeting desired marbling where diet is controlled according to various parameters in an effort to achieve the desired marbling. As to milk production, whether for dairy or nursing, a method can include targeting a desired quantity and/or quality of milk where diet is controlled according to various parameters in an effort to achieve the desired milk characteristics.

As an example, for humans that compete in body building, a diet may be prescribed pre-competition for fat removal, followed by a post-competition state, which may be a recovery or a training state.

As an example, a retirement or assisted care facility may utilize equipment and techniques to help maintain or improve health of residents. As an example, in the elderly, a goal may be to increase muscle mass and muscle strength.

As explained, one kilogram of fat is a substantial amount of energy, particularly when compared to one kilogram of muscle, which includes a substantial amount of water. For pure fat, energy is approximately 9 calories per gram while, for dry protein from muscle, energy is 4 calories per gram. As an example, a user may experience a relatively constant weight where various metabolic processes occur such as losing fat and gaining muscle, which may be beneficial.

As an example, a method can include using water fraction as a variable. For example, consider water fraction for muscle and/or for fat. As to energy, a method may take into account a water fraction. For example, consider one or more approaches as explained in DiGirolamo et al. pertaining to water fraction in adipose tissue.

As an example, a method can include predicting an outcome or probable outcome for a user if that user ingests a particular type of food. For example, consider eating 1 gram of protein and outputting what will happen to the user (e.g., that 1 gram of protein). As an example, a method can include outputting what is gained and/or what is lost. As an example, consider some protein may become fat.

As an example, an app can be executable using a device, a system, etc., where it can include features for health-care/lifestyle industry (e.g., weight control, diet & fitness, etc.). As an example, such an app may promote one or more ways to lose weight (e.g., eat proteins and train to preserve muscles, and lose mostly fat tissue, which can be in contrast to eat only fruits without exercise, and lose mostly muscle mass, which may give rise to rapid weight loss, but be unhealthy). As explained, an approach may utilize a formula that can determine DE, which, for example, consider lose more fat than muscles provides a high DE. As an example, a DE may be based on three measurements (e.g., three measured values). As an example, DE calculation circuitry can be included in a system, which can include one or more sensors, one or more user interfaces, etc. Such a system can provide actionable insights based on physics and available inputs, where physics may be via a physics-based approach that is implemented using a physics-based model, an empirical model and/or a trained machine model.

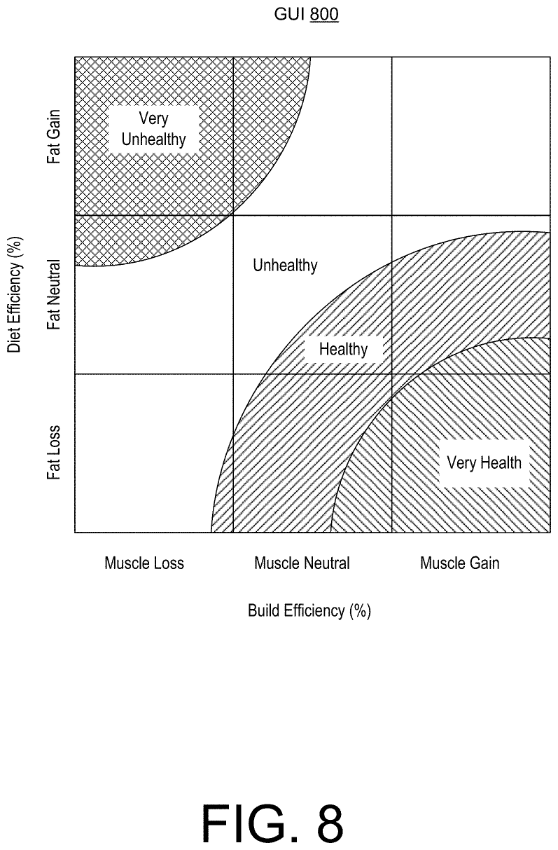

FIG. 8 shows an example of a GUI 800 that includes an area that represents DE and build efficiency (BE). As shown, the DE direction can correspond to a scale from fat loss to fat neutral to fat gain while the BE direction can correspond to a scale from muscle loss to muscle neutral to muscle gain. As shown, the scales can define nine regions within the area. Using the various metrics, sub-areas can be defined for an individual such as, for example, very unhealthy, unhealthy, healthy and very healthy. As an example, a device and/or a system can generate such sub-areas and, for example, one or more indicators that show where an individual currently is as to DE and BE, historical DE and/or BE and desirable DE and BE (e.g., direction, direction with respect to time, etc.). For example, a device and/or a system may be predictive and provide a trajectory that spans a week, a month, a year, etc., as to where an individual may be headed based on data. Such an approach can allow the individual to determine changes that may be beneficial and/or otherwise desirable.

As an example, a GUI may be rendered in color with colors that are indicative of regions, sub-areas, progress, etc. As an example, a GUI may be dynamic and/or interactive. As to colors, a spectrum may be utilized. As an example, red and green may be utilized where green is deemed good and red is deemed bad, optionally with yellow as an intermediate color (e.g., caution, etc.).

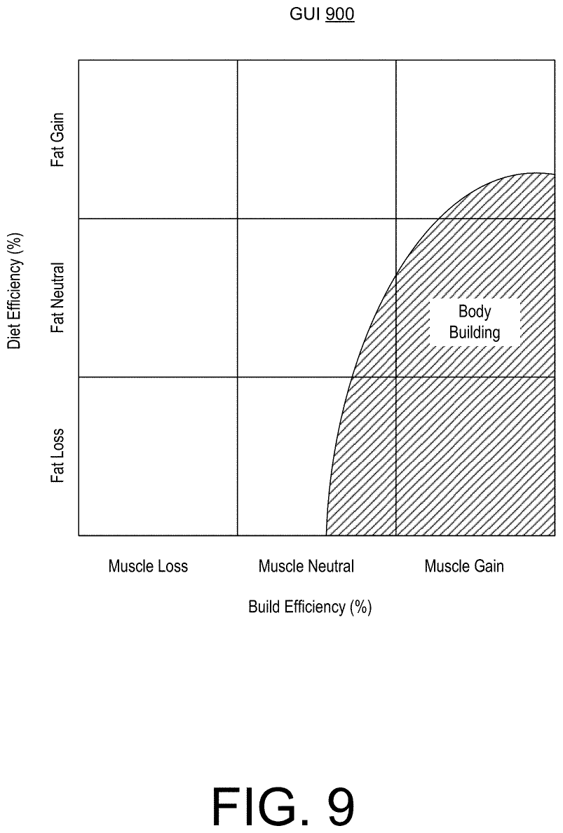

FIG. 9 shows an example of a GUI 900 that can be generated for a particular purpose such as body building, where an individual may desire gaining muscle mass.

As an example, a GUI may be utilized for a number of individuals. For example, consider a training coach that aims to track individual team members or clients. In such an example, multiple indicators may be rendered to show how the team members are progressing with respect to DE and/or BE. In such an approach, each individual may be assessed relative to one or more others. In a relative approach, the training coach can become aware of what individuals are excelling and what individuals are in need of further guidance and/or changes.

While a training coach is given as an example, other examples of groups can be patient groups, family groups, work groups, military groups, etc. Information rendered may be useful for procurement as to food items where a cafeteria, group meals, individual meals, etc., are to be prepared.

As an example, one or more GUIs may be suitable for use by a grocery store that can aim to order food that is beneficial toward health of customers.

As an example, one or more GUIs may be suitable for use by the insurance industry, where, for example, one or more actuaries may be involved in assessing risk and/or ways to promote health.

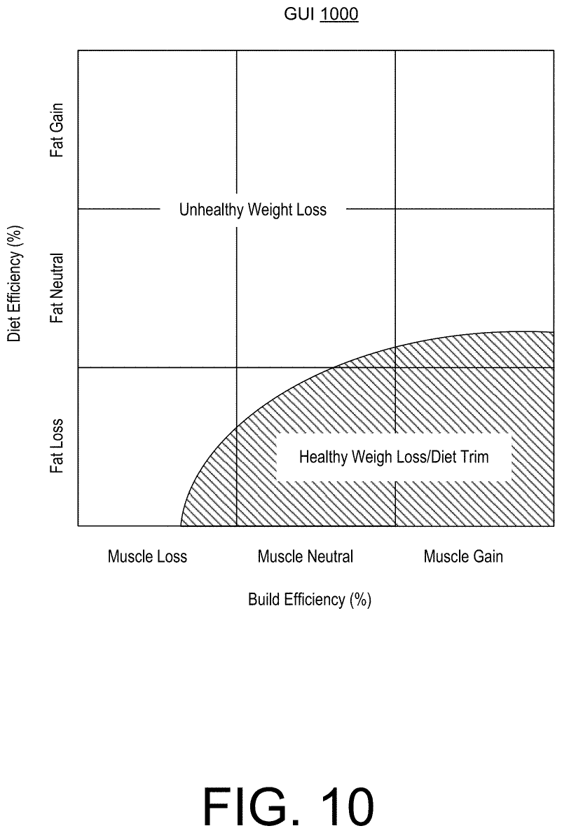

FIG. 10 shows an example of a GUI 1000 that includes a region that is for healthy weight loss and/or a trimming diet. As explained, one or more types of GUIs may be generated for rendering to one or more displays.

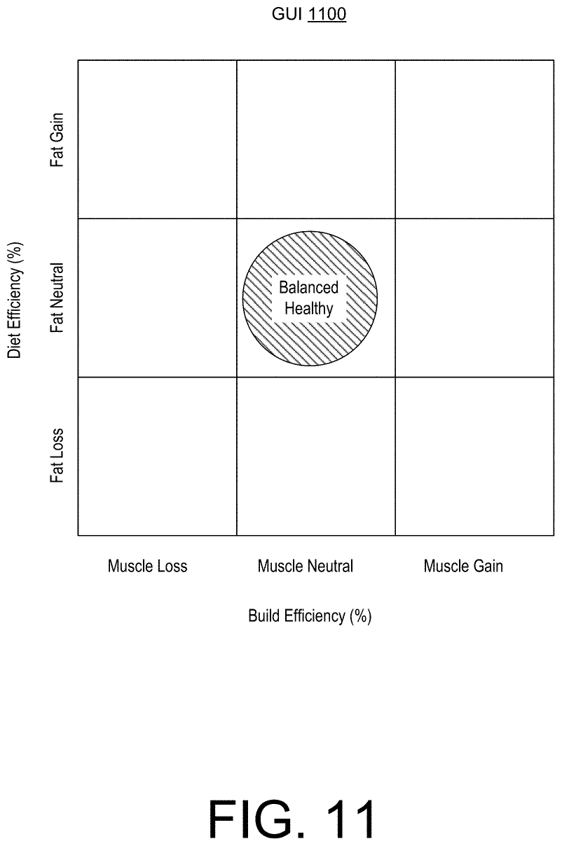

FIG. 11 shows an example of a GUI 1100 that includes a region that is for a balanced healthy sub-area. As explained, one or more types of GUIs may be generated for rendering to one or more displays.

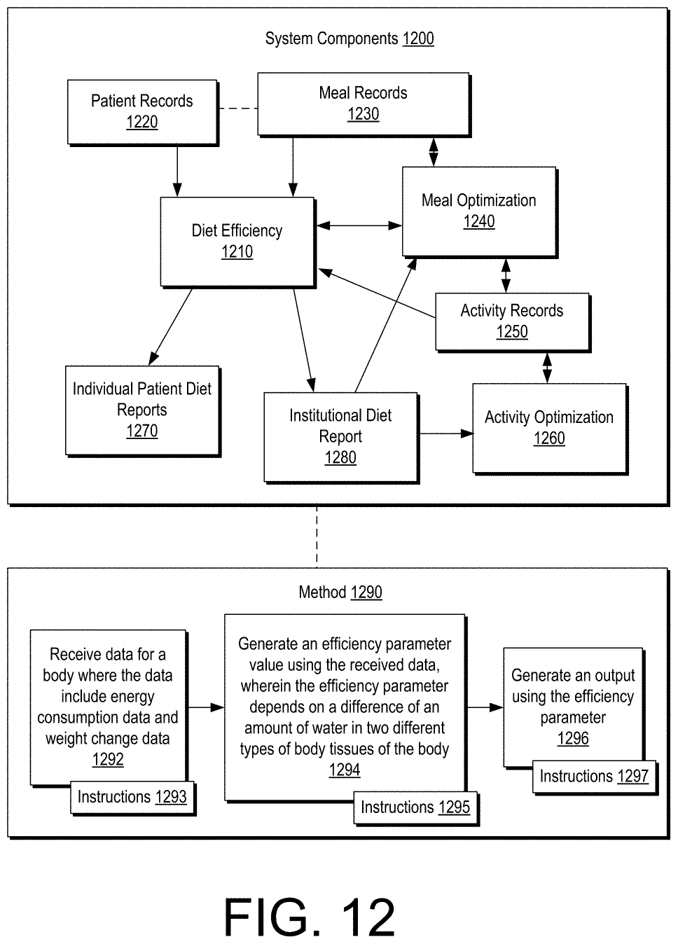

FIG. 12 shows various examples of system components 1200 and an example of a method 1290. One or more of such system components may be utilized for interactions with a population of living organisms, for example, out of a shared food supply system (e.g., hospitals, prisons, retirement homes, schools, military camps, farming of animals, etc.). Information about accommodated organisms can be maintained in a patient record component 1220 (e.g., database(s)). As an example, together with information about food supply in a meal records component 1230 (e.g., database(s)), a system can compute DE using the DE component 1210 for one or more organisms, which may be computed simultaneously, individually, etc. The impact of diet can be reported on an individual level per a component 1270 or, for example, on a sub-system level per a component 1280 (e.g., a department, a hospital wing, a barrack, etc.). Based on such information, and one or more objectives, a food plan can be updated in order to better meet one or more objectives, which may utilize a meal optimization component 1240. Such an approach can optionally be combined with one or more activity records per a component 1250 (e.g., exercise regime conducted, etc.) and, for example, combined with activity optimization per a component 1260, which can seek an activity plan to better meet one or more objectives. One or more of the system components 1200 may provide for GUI interactions (e.g., in a client-server architecture, a device-to-device architecture, etc.) where an individual and/or a responsible individual (e.g., a doctor, a trainer, a supply manager, a researcher, an animal caretaker, etc.) can visual and interact with one or more of the system components 1200, which may provide for messaging, alerts, etc., to help further an objective, control activities, control food supply, control food preparation, etc. As an example, one or more GUIs such as the GUI 700 may be utilized to establish one or more relationships between biological processes and data, which can include metrics such as DE. As an example, DE can be indicative of one or more biological processes, which, in turn, can be indicative of health and/or progress toward an objective.

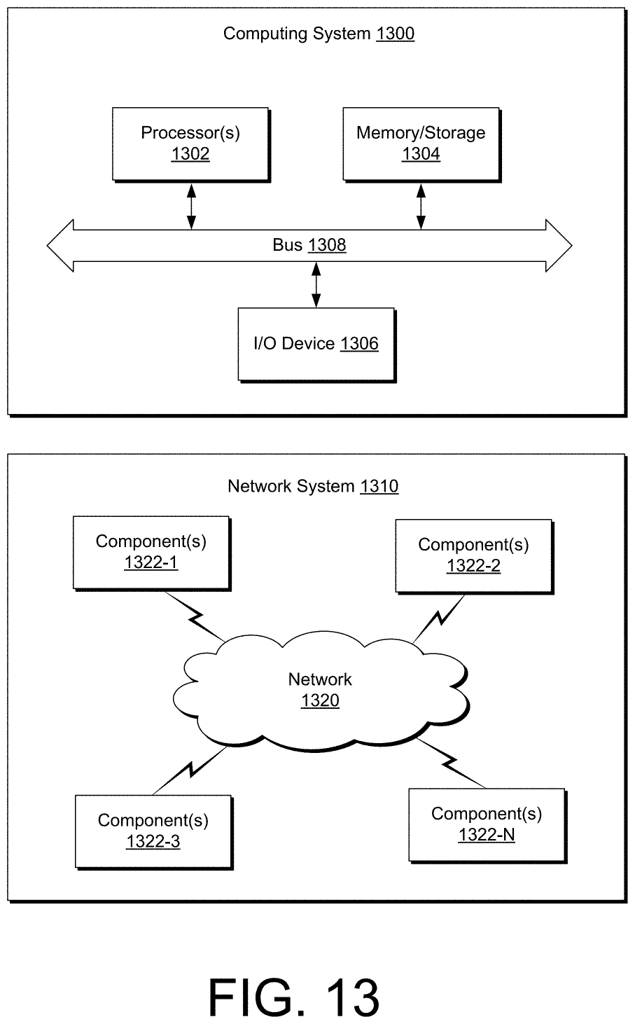

As shown in FIG. 12, the example method 1290 can include a reception block 1292 for receiving data for a body where the data include energy consumption data and weight change data; a generation block 1294 for generating an efficiency parameter value using the received data, where the efficiency parameter depends on a difference of an amount of water in two different types of body tissues of the body; and a generation block 1296 for generating an output using the efficiency parameter. In such an example, the method can include receiving additional data, generating another efficiency parameter value, and comparing the efficiency parameter values to determine a trend. Such a method may be implemented using a computing system, which can include, for example, one or more of the system components 1200 and/or one or more components as shown in the FIGS. 1, 2, 4, 5 and 13 where output may be, for example, output such as in one or more of FIGS. 4, 5, 6, 7, 8, 9, 10, and 11. FIG. 12 also shows instruction blocks 1293, 1295 and 1297, which can be computer-readable storage media instructions (e.g., a computer-program product, etc.), which can be stored in a non-transitory medium or media that is not a carrier wave nor a signal and executable by one or more processors, etc. For example, the computing system 1300 of FIG. 13 (e.g., and/or the device 190 of FIG. 1, etc.), can include such instructions for performing a method, a portion of a method, etc.

As an example, a method can include receiving data for a body where the data include energy consumption data and weight change data; generating an efficiency parameter value using the received data, where the efficiency parameter depends on a difference of an amount of water in two different types of body tissues of the body; and generating an output using the efficiency parameter. In such an example, the method can include receiving additional data, generating another efficiency parameter value, and comparing the efficiency parameter values to determine a trend.

As an example, a method can include receiving data that includes receiving data via a device that includes a weight sensor, receiving data via a caloric database and/or receiving data via a user interface of a device.

As an example, a method can include generating output that includes rendering a graphic to a display or, for example, consider a biofeedback approach where a signal is issued via a transducer to a user. Such a signal may be an electrical stimulation signal, an audio signal, etc. For example, consider a method whereby a user notifies an app that a meal is about to occur. The user may input the items to be eaten in the app where the app may cause a device to issue a signal as to one or more of the items, which may be a signal that the item is OK or not OK for a particular diet. In such an example, the signal may be discrete such that others are not aware of the feedback. For example, consider a watch as a wearable where an electrical stimulation, a haptic signal, etc., can be issued to notify the wearer in a discrete manner. In such an approach, the user's company or others may not be aware of what the user is doing and being instructed to do. In such an approach, the user may maintain a social appearance that is not distracting to others. For example, merely playing with a smart watch or a smart phone at meal time has become a relatively common occurrence, which some may find acceptable. For those that do, the user may enter items and discretely receive feedback while the others consider the user to be merely playing with his smart watch or smart phone. Such an approach may make the technology as to use and feedback more acceptable and hence easier to follow (e.g., conceal to others that he is on a dieting program).

As an example, an application can include one or more application programming interfaces (APIs) that can receive one or more types of API calls. For example, consider a grocery shopping application, a menu application, etc., that can make one or more API calls to an application that pertains to one or more diets of one or more users. In such an example, the calling application can transmit and/or receive information pertaining to one or more diets. In such an example, the calling application may make one or more decisions using such information such as, for example, recommending and/or organizing a grocery list for a number of meals for a number of people, recommending and/or organizing menu items for one or more sources of such items (e.g., a restaurant, restaurants, etc.), etc. As an example, a calling application may provide guidance as to how much of a food stock to buy and/or what to supplement it with and optionally how much. Such an approach can provide for meal planning aligned with diet objectives. As to an example of a calling application, consider a GRUBHUB application, that can be for online and mobile prepared food ordering and delivery that connects diners with local takeout restaurants. As another example, consider WALMART Store Pick-up, as an ordering application that can allow a user to order items for quick pick-up at a store or for delivery. As another example, consider PIZZAHUT for ordering and pick-up or delivery. In such examples, items may be recommended, organized, etc., using one or more values provided via an API call to a diet application. In such examples, data as to content of items may be utilized, for example, to determine how DE may be impacted such that DE may be optimized via recommendation of one or more items. As mentioned, one or more other applications may be tied-in, such as, for example, a fitness tracking application, etc. As an example, one or more of a health tracking application, a health insurance tracking application, etc., may be tied-in. As an example, consider a medical records tie-in where, for example, particular foods are to be avoided due to one or more medical conditions (e.g., allergies, arthritis, etc.).

As an example, an approach may operate in a reverse direction where a diet application calls a grocery shopping application, a menu application, etc. In such an approach, the diet application may facilitate ordering an appropriate amount of food, appropriate types of food, etc., which may be fit within a schedule (e.g., daily, weekly, monthly, etc.). As an example, an API may include a call for DE and/or a call for one or more values that are based at least in part on DE.

As to various types of animals, a feedback mechanism may be within acceptable guidelines as to the type and strength of the feedback. For example, consider a bark-collar of a dog, an electric fence, etc., where electrical stimulation may be of an acceptable level to deter a behavior. As to livestock, one or more types of mechanisms may be utilized to determine a change in weight during a feeding and/or drinking session where a feedback signal is issued to an animal or animals. For example, consider a sensor, which may be a wearable, that can determine how much mass an animal consumed from a common trough where that animal receives a feedback signal that deters the animal from continuing to eat (e.g., to distract the animal from continuing to feed, etc.). As an example, where a trough is mechanized, it may take action in response, for example, such as draining feed from a trap door such that the trough becomes empty, positioning a cover, etc.

As an example, a method can include receiving data, generating an efficiency parameter value, and generating output where such actions are performed by circuitry of a weight scale, by circuitry of a wearable computing device, and/or by circuitry of a smart phone.

As an example, a method can include transmitting output via a network interface to a computing system where the computing system utilizes the output to train a predictive machine learning model.

As an example, a method can include transmitting output via a network to a computing system and, in response, receiving a prediction as generated by a trained predictive machine learning model.

As an example, a method can pertain to two different types of body tissues of the body, which can be muscle tissue and fat tissue. As an example, a method can include adjusting one or more variables for the two different types of body tissues of the body.

As an example, a method can include generating a hydration indicator using at least an efficiency parameter value. As an example, a body can be subject to a diet and where an efficiency parameter value is indicative of an efficiency of the diet.

As an example, a system can include a processor; memory operatively coupled to the processor; and processor-executable instructions stored in the memory to instruct the system, the instructions including instructions to: receive data for a body where the data include energy consumption data and weight change data; generate an efficiency parameter value using the received data, where the efficiency parameter depends on a difference of an amount of water in two different types of body tissues of the body; and generate an output using the efficiency parameter.

As an example, one or more computer-readable storage media can include computer-executable instructions to instruct a computer, the instructions including instructions to: receive data for a body where the data include energy consumption data and weight change data; generate an efficiency parameter value using the received data, where the efficiency parameter depends on a difference of an amount of water in two different types of body tissues of the body; and generate an output using the efficiency parameter. As an example, a computer program product can include computer-executable instructions to instruct a computing system to perform a method such as one or more of the methods described herein.

FIG. 13 shows components of an example of a computing system 1300 and an example of a networked system 1310, either of which may be utilized in one or more systems, methods, etc., as described herein. The system 1300 includes one or more processors 1302, memory and/or storage components 1304, one or more input and/or output devices 1306 and a bus 1308. In an example embodiment, instructions may be stored in one or more computer-readable media (e.g., memory/storage components 1304). Such instructions may be read by one or more processors (e.g., the processor(s) 1302) via a communication bus (e.g., the bus 1308), which may be wired or wireless. The one or more processors may execute such instructions to implement (wholly or in part) one or more attributes (e.g., as part of a method). A user may view output from and interact with a process via an I/O device (e.g., the device 1306). In an example embodiment, a computer-readable medium may be a storage component such as a physical memory storage device, for example, a chip, a chip on a package, a memory card, etc. (e.g., a computer-readable storage medium).

In an example embodiment, components may be distributed, such as in the network system 1310 with a network 1320. The network system 1310 includes a network 1320 and components 1322-1, 1322-2, 1322-3, . . . 1322-N. For example, the components 1322-1 may include the processor(s) 1302 while the component(s) 1322-3 may include memory accessible by the processor(s) 1302. Further, the component(s) 1302-2 may include an I/O device for display and optionally interaction with a method. The network may be or include the Internet, an intranet, a cellular network, a satellite network, etc.

As an example, a device may be a mobile device that includes one or more network interfaces for communication of information. For example, a mobile device may include a wireless network interface (e.g., operable via IEEE 802.11, ETSI GSM, 5G, BLUETOOTH, satellite, etc.). As an example, a mobile device may include components such as a main processor, memory, a display, display graphics circuitry (e.g., optionally including touch and gesture circuitry), a SIM slot, audio/video circuitry, motion processing circuitry (e.g., accelerometer, gyroscope), wireless LAN circuitry, smart card circuitry, transmitter circuitry, GPS circuitry, and a battery. As an example, a mobile device may be configured as a cell phone, a tablet, etc. As an example, a method may be implemented (e.g., wholly or in part) using a mobile device. As an example, a system may include one or more mobile devices.

As an example, a system may be a distributed environment, for example, a so-called “cloud” environment where various devices, components, etc. interact for purposes of data storage, communications, computing, etc. As an example, a device or a system may include one or more components for communication of information via one or more of the Internet (e.g., where communication occurs via one or more Internet protocols), a cellular network, a satellite network, etc. As an example, a method may be implemented in a distributed environment (e.g., wholly or in part as a cloud-based service).

As an example, information may be input from a display (e.g., consider a touchscreen), output to a display or both. As an example, information may be output to a projector, a laser device, a printer, etc. such that the information may be viewed. As an example, information may be output stereographically or holographically. As to a printer, consider a 2D or a 3D printer.

Although only a few example embodiments have been described in detail above, those skilled in the art will readily appreciate that many modifications are possible in the example embodiments. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed is:

1. A method comprising:
receiving data for a body wherein the data comprise energy consumption data and weight change data;
generating an efficiency parameter value using the received data, wherein the efficiency parameter depends on a difference of an amount of water in two different types of body tissues of the body; and
generating an output using the efficiency parameter.

2. The method of claim 1 comprising receiving additional data, generating another efficiency parameter value, and comparing the efficiency parameter values to determine a trend.

3. The method of claim 1 wherein receiving data comprises receiving data via a device that comprises a weight sensor.

4. The method of claim 1 wherein generating the output comprises rendering a graphic to a display.

5. The method of claim 1 wherein receiving data, generating the efficiency parameter value, and generating the output are performed by circuitry of a weight scale.

6. The method of claim 1 wherein receiving data, generating the efficiency parameter value, and generating the output are performed by circuitry of a wearable computing device.

7. The method of claim 1 wherein receiving data, generating the efficiency parameter value, and generating the output are performed by circuitry of a smart phone.

8. The method of claim 1 comprising transmitting the output via a network interface to a computing system wherein the computing system utilizes the output to train a predictive machine learning model.

9. The method of claim 1 comprising transmitting the output via a network to a computing system and, in response, receiving a prediction as generated by a trained predictive machine learning model.

10. The method of claim 1 wherein the two different types of body tissues of the body comprise muscle tissue and fat tissue.

11. The method of claim 1 comprising adjusting one or more variables for the two different types of body tissues of the body.

12. The method of claim 1 comprising generating a hydration indicator using at least the efficiency parameter value.

13. The method of claim 1 wherein the body is subject to a diet and wherein the efficiency parameter value is indicative of an efficiency of the diet.

14. The method of claim 1 comprising generating a graphic renderable via a graphical user interface wherein the graphic comprises a diet efficiency dimension and a build efficiency dimension.

15. The method of claim 14 wherein the diet efficiency dimension is defined at least in part with respect to fat loss and fat gain and wherein the build efficiency dimension is defined at least in part with respect to muscle loss and muscle gain.

16. The method of claim 14 wherein the graphic comprises at least one region representative of a state of a health of the body.

17. The method of claim 14 wherein the graphic comprises an unhealthy weight loss region and a healthy weight loss region.

18. A system comprising:
a processor;
memory operatively coupled to the processor; and
processor-executable instructions stored in the memory to instruct the system, the instructions comprising instructions to:
receive data for a body wherein the data comprise energy consumption data and weight change data;
generate an efficiency parameter value using the received data, wherein the efficiency parameter depends on a difference of an amount of water in two different types of body tissues of the body; and
generate an output using the efficiency parameter.

19. One or more computer-readable media comprising processor-executable instructions executable to cause a system to:
receive data for a body wherein the data comprise energy consumption data and weight change data;
generate an efficiency parameter value using the received data, wherein the efficiency parameter depends on a difference of an amount of water in two different types of body tissues of the body; and
generate an output using the efficiency parameter.
